(19) United States
(12) Patent Application Publication
(10) Pub. No.: US 2021/0244530 A1
(43) Pub. Date: Aug. 12, 2021

(54) DEVICE FOR PROVIDING AND INSERTING A TRANSPLANT OR IMPLANT INTO A HUMAN OR ANIMAL BODY AND READY-TO-USE SET COMPRISING SAID DEVICE

(71) Applicant: GEUDER AG, Heidelberg (DE)

(72) Inventors: Hamadi EL-AYARI, Frankfurt (DE); Juergen EDER, Gaiberg (DE)

(21) Appl. No.: 16/972,851

(22) PCT Filed: Jun. 4, 2019
(86) PCT No.: PCT/DE2019/200060

§ 371 Date: Dec. 7, 2020

(30) Foreign Application Priority Data
Jun. 8, 2018 (DE) 10 2018 209 194.3

(51) Int. Cl. A61F 2/14; A61F 2/00

(52) U.S. Cl. CPC A61F 2/148; A61F 2250/0081; A61F 2/0095

(57) ABSTRACT

The invention relates to a device for providing and inserting a transplant or implant into a human or animal body, more particularly for ophthalmological operations, preferably for performing a lamellar keratoplasty, such as a DMEK operation, comprising a cartridge (1) or sleeve, which has a holding region (2) for the transplant or implant and two opposite end openings (3, 4) each fluidically connected to the holding region (2), wherein the first opening (3) has a larger diameter than the second opening (4) and wherein, in order to produce a fluidic connection between the holding region (2) and a syringe (5), both openings (3, 4) can be coupled to the syringe (5) directly or by means of a connecting apparatus. With a view to simply and safely providing and inserting a transplant or implant into the body by simply designed means, the device is designed and further developed in such a way that the cartridge (1) or sleeve is mounted in a holder (6) and that the holder (6) has a pivoting apparatus (7) for pivoting the cartridge (1) or sleeve about a pivot axis (8) into a coupling position for coupling at least one of the two openings (3, 4) to the syringe (5). The invention further relates to a ready-to-use set comprising a device as described above in a sterile packaging.

### CROSS REFERENCE TO RELATED APPLICATIONS

This application is a national stage application, filed under 35 U.S.C. § 371, of International Application No. PCT/DE2019/200060, filed Jun. 4, 2019, which claims priority to Germany Application No. 102018209194.3, filed Jun. 8, 2018, the contents of both of which as are hereby incorporated by reference in their entirety.

### BACKGROUND

### Technical Field

The invention relates to a device for providing and inserting a transplant or an implant into a human or animal body, in particular for ophthalmological interventions, preferably for carrying out a lamellar keratoplasty, for example, a DMEK (Descemet's Membrane Endothelial Keratoplasty) operation, having a cartridge or sleeve which has a holding region for the transplant or implant and two opposite end openings, each fluidically coupled to the holding region, wherein the first opening has a larger diameter than the second opening and wherein both openings can be coupled to a syringe directly or by means of a connecting apparatus to form a fluidic connection between the holding region and the syringe.

Furthermore, the present invention relates to a ready-to-use set comprising such a device in a sterile package.

### Description of Related Art

The transplantation of the human cornea belongs to the commonest and most successful transplant surgery interventions performed. The transplantation of the cornea using all its layers (epithelium, Bowman's membrane, stroma, Descemet's membrane and endothelial cell layer) in the context of a perforating keratoplasty has been known for more than 100 years. Using this operation method, good results are generally achieved, although the convalescence is very slow and a recovery of vision can normally only finally be achieved after removal of the second suture. This can involve a timespan of up to 18 months after the intervention. Additionally, due to the surgically induced astigmatism, this operating method does not always lead to a satisfactory post-operative refractive result.

Well over half of all corneal transplantations are carried out due to diseases of the corneal endothelium. For these diseases, in principle, a layer-specific replacement of the cornea would be suitable. Suitable techniques now exist for this. With the transplantation of the Descemet's membrane with an attached stroma portion (DSAEK), for the last several years, a much faster vision recovery, a significantly reduced surgically induced astigmatism and consequently improved patient satisfaction have been achieved, as compared with perforating keratoplasty.

Conventional perforating keratoplasty for endothelial corneal diseases is now often regarded as overtreatment, and the transplantation of vital endothelial cells alone is usually considered sufficient. Therefore, posterior lamellar techniques, in particular DMEK (Descemet's Membrane Endothelial Keratoplasty) have now become well established as largely atraumatic alternatives for the treatment of endothelial corneal diseases. DMEK is limited to the transplantation of the isolated Descemet's membrane with the endothelial cells located thereon. The transplant no longer contains any stromal constituents and characteristically has a thickness of only approximately 15 μm.

From DE 10 2010 051 458 A1, there is known a device for providing and inserting a transplant or implant into a human or animal body, wherein the device has a cartridge or sleeve which has a holding region for the transplant or implant and two opposite end openings, each fluidically connected to the holding region. The first opening has a larger diameter than the second opening and, in order to form a fluidic connection between the holding region and a syringe, both openings can be coupled to the syringe directly or by means of a connecting apparatus. In other words, the cartridge or sleeve can be coupled interchangeably with one of the two openings to a syringe directly or via a connecting apparatus.

With the known device, it is usual, in particular when applying the DMEK, to use three components in the form of the cartridge or sleeve, a syringe and a connecting hose serving as a connecting apparatus between the cartridge or the sleeve and the syringe. This use of a system consisting of three components has proved in practice to be impracticable. Firstly, in principle, a complex assembly of these three components is necessary before use. Secondly, depending on whether the cartridge or sleeve is used for drawing a transplant or implant into the holding region or for injecting the transplant or implant, a complex re-plugging of the cartridge or sleeve to the connecting hose must take place. For example, a surgeon must concentrate intensively during the performance of a DMEK and therewith an injection of a transplant into an anterior chamber of an eye, since the transplant must reach the anterior space of the anterior ocular chamber, between the iris and the cornea in a highly controlled manner. A rotation of the transplant roll is to be prevented so that a condition of the transplant is maintained in that the Descemet's membrane faces upwardly in the direction of the cornea and the endothelial cell layer faces downwardly in the direction of the iris—since otherwise the unfolded transplant must be rotated intraoperatively in the anterior chamber, if possible without touching it—in order to prevent endothelial cell loss. Furthermore, a central positioning of the transplant and prevention of accidental flushing out through the incision are essential. This is difficult with the existing system, since ultimately the syringe for fluidic injection must be operated with both hands and simultaneously, the cartridge or sleeve which is inserted in the incision in the eye must be operated and possibly repositioned. In this process, at least a precise holding and possibly a rotation is required if the transplant roll rotates in an undesirable manner.

Furthermore, in the conventional connecting hose, both when drawing in the transplant or implant and also during injection of the transplant or implant, an additional liquid volume is always moved, which is actually not needed for reliable injection of a transplant or implant, but which merely creates the connection between the cartridge or sleeve and the syringe and the liquids introduced therein. This additional liquid volume can lead to additional turbulences which are extremely undesirable in the context of the reliable and controlled positioning of the transplant in the anterior ocular chamber.

Furthermore, with the known system consisting of the three above-mentioned components, it can be problematic that due to reflection of light on the shiny glass surface of the commonly used cartridge or sleeve—depending on the light incidence angle and the ambient lighting in an operating room—the view of the transplant or implant can be hindered or impaired, which presents a further disadvantage of the known system. Even the customary staining of a transplant or implant to increase its visibility does not significantly further assist in the event of undesirable reflections. The visual checking of the position of the transplant or implant roll in the cartridge or sleeve—at any time point of the implantation or injection—is, however, of great importance, so that an already injected roll does not subsequently have to be turned in the eye, without contact, in order to have the Descemet's membrane arranged above and the endothelial cell layer arranged underneath. The right position of the transplant or implant is therefore of great importance for a successful operation and, if needed, can be corrected by turning or adjusting the cartridge or sleeve during the injection procedure, provided the position of the transplant or implant in the cartridge or sleeve can be known exactly, although reflections which occur are highly disadvantageous.

### BRIEF SUMMARY

It is therefore an object of the present invention to provide a device for providing and inserting a transplant or implant into a human or animal body and a ready-to-use set with such a device, according to which a simple and reliable provision and insertion of a transplant or implant into a human or animal body is enabled with simply designed means.

According to the invention, the above object is achieved by means of a device and a ready-to-use set according to the claims presented herein. Thereby, the device according to the invention is configured and developed such that the cartridge or sleeve is mounted in a holder and that the holder has a pivoting apparatus for pivoting the cartridge or sleeve about a pivot axis into a coupling position of at least one of the two openings with the syringe.

In a manner according to the invention, it was firstly discovered that a simple and reliable introduction of a transplant or implant into a human or animal body does not necessarily have to take place with the conventionally usual system consisting of three components in the form of a cartridge or sleeve, a connecting hose and a syringe. In a further manner according to the invention, it was then discovered that with skillful mounting of the cartridge or sleeve in a holder, the above-mentioned object is achieved in a surprising manner. For this purpose, according to the invention, the cartridge or sleeve is mounted quite specifically in a holder, whereby the holder has a pivoting apparatus for pivoting the cartridge or sleeve about a pivot axis. The pivoting of the cartridge or sleeve takes place in such a way that the cartridge or sleeve is brought into a—directly or, via a connecting apparatus, indirectly—coupling position of at least one of the two openings with the syringe. The coupling of the cartridge or sleeve in a suitable coupling position is very significantly simplified by the mounting according to the invention of the cartridge or sleeve in a holder with a pivoting apparatus, whereby the pivoting of the cartridge or sleeve is typically possible—even one-handedly—by grasping the holder and actuating the pivoting apparatus. Following the desired pivoting of the cartridge or sleeve into a suitable coupling position, a coupling with the syringe—directly, or indirectly via a connecting apparatus—can take place in a simple and reliable manner. Incorrect operations are practically precluded.

Consequently, with the device according to the invention and also a corresponding ready-to-use set with such a device, a simple and reliable provision and insertion of a transplant or implant into a human or animal body is enabled with simply designed means.

With regard to a particularly simple and reliable provision and insertion of a transplant or implant, the pivoting apparatus can be configured for pivoting the cartridge or sleeve from a first coupling position for the first opening, depending on the geometry of the cartridge or sleeve—with a straight, elongate geometry (both openings diametrically opposite)—preferably by 180°, about the pivot axis into a second coupling position for the second opening and vice versa. In another embodiment of the device, a pivoting by 120° or another angle could be sufficient to change between the first and second coupling position. The pivoting apparatus thereby provides a pivoting possibility for the cartridge or sleeve between the two utilization positions of the cartridge or sleeve, firstly, for drawing in a transplant or implant and, secondly, for injecting or inserting a transplant or implant. Thereby, both the provision and also the insertion of a transplant or implant is enabled, depending on the coupling position selected for the first or second opening.

With regard to a further simplification and increase in the reliability on provision and insertion of a transplant or implant, the pivoting apparatus can have a snap-in locking device for locking, latching or detent of a pivoting movement of the cartridge or sleeve in at least one of the two coupling positions. As soon as the cartridge or sleeve has reached at least one of the two coupling positions by means of the pivoting, the locking, latching or detent of the cartridge or sleeve takes place for fixing the cartridge or sleeve in the first and/or second coupling position. The snap-in locking device can have suitable grooves in conjunction with locking noses—each, for example, in surfaces or components of the pivoting apparatus sliding over one another during the pivoting—which, on reaching the first and/or second coupling position, engage with one another for locking, latching or detent of the pivoting movement.

With regard to a simple achievement of at least one of the two coupling positions of the cartridge or sleeve, the pivoting apparatus can have an actuating apparatus for pivoting the cartridge or sleeve. In a particularly simple manner, such an actuating apparatus can have a lever or a wheel.

In order to ensure a reliable mounting of the cartridge or sleeve during use of the device, the pivoting apparatus can have a fixing apparatus for holding the cartridge or sleeve during the pivoting. By this means, a reliable positioning of the cartridge or sleeve in a desired coupling position is enabled with a high degree of safety. Such a fixing apparatus can have, for example, a sleeve or bracket for preferably completely grasping the cartridge or sleeve. By this means, a particularly secure fixing of the cartridge or sleeve during the pivoting is achievable.

Furthermore, with regard to a particularly simple and reliable provision and insertion of a transplant or implant, the holder can have a holding apparatus for a syringe, said holding apparatus being movable back and forth relative to the pivoting apparatus by means of a guide. After reaching a desired coupling position through the pivoting of the cartridge or sleeve by means of the pivoting apparatus, the holding apparatus for a syringe can be moved toward the pivoting apparatus, so that the coupling between the cartridge or sleeve and the syringe can be carried out—with this axial movement process, also, a locking or latching into the end positions can be realized. During the movement of the holding apparatus relative to the pivoting apparatus, the direct or indirect coupling takes place between the cartridge or sleeve with the syringe or the formation of the fluidic connection between the holding region of the cartridge or sleeve and the syringe. In order to swap the coupling position of the cartridge or sleeve, the holding apparatus can be moved away relative to the pivoting apparatus by means of the guide, in order to enable a free pivoting of the cartridge or sleeve in the state uncoupled from the syringe. In this way, swapping can take place between the coupling of the syringe with the first opening and with the second opening of the cartridge or sleeve. It should be noted that the movement between the pivoting apparatus and the holding apparatus involves a movement of these two components relative to one another. Both components are associated with the holder or arranged thereon and either the pivoting apparatus can be moved in the direction of the holding apparatus or away therefrom, or the holding apparatus can be moved toward or away from the pivoting apparatus or both the pivoting apparatus and also the holding apparatus can be moved toward or away from one another, in order to realize the desired relative movement.

In an advantageous manner, the guide can have a preferably sled-like linear guide. This linear guide can have at least one rail configured fixed to the pivoting apparatus or to the holding apparatus, along which a sliding element is displaceable back and forth, wherein the sliding element can be coupled to syringe or the pivoting apparatus in order to be able to move the syringe relative to the pivoting apparatus and thus relative to the cartridge or sleeve. The sliding element can be coupled—if the at least one rail is configured fixed to the pivoting apparatus—to the holding apparatus for the syringe or such a holding apparatus for a syringe. In this way, a particularly compact configuration of the device can be realized. Alternatively, the guide can have a preferably bayonet-like twist guide which ensures the relative movement between the pivoting apparatus and the holding apparatus for the syringe. Such a twist guide provides, firstly, a linear component of the relative movement between the pivoting apparatus and the holding apparatus and, secondly, a rotation component about the direction of the linear component. Preferably, one end position of this twist-slide guide forms a coupling position between the cartridge or sleeve, and another end position forms a non-coupled position in which the cartridge or sleeve can be pivoted by means of the pivoting apparatus. It should again be noted at this point that a coupling can take place between the syringe and the cartridge or sleeve, both directly as well as via a connecting apparatus. In this regard, these two possibilities of the coupling—direct or indirect—are always included wherever a coupling between the cartridge or the sleeve and the syringe is mentioned in this document.

Furthermore, with regard to a particularly simple and reliable provision and insertion of a transplant or implant into a body, the holding apparatus can have a fixing apparatus at least in parts encompassing a cylindrical region of the syringe, preferably a sleeve or bracket. By means of such a fixing apparatus, a secure coupling of a syringe to the holder of the device is enabled. A reliable overall function of the device is, in particular, thereby ensured.

Alternatively or in addition to such a fixing apparatus, the holding apparatus can have an adapter operating as a connecting apparatus with a cut-out or a coupling region for the syringe and preferably a nozzle extending at the end side out of the cut-out or out of the coupling apparatus for coupling to the cartridge or sleeve. In this case, the coupling does not take place directly between the syringe and the cartridge or sleeve, but rather indirectly via an adapter acting as a connecting apparatus. For the secure coupling of a syringe, the adapter has a cut-out or a coupling region for the syringe. In order to ensure a fluidic connection between the cut-out region of the cartridge or sleeve and the syringe, the adapter preferably has a nozzle extending out of the cut-out or out of the coupling region, with which the cartridge or sleeve can be coupled. In this case, the syringe is insertable into the cut-out, whereby the cut-out can have a hollow space that is substantially cylindrical or adapted to the external form of the syringe, in order to ensure a secure and exactly fitting holding of the syringe in the cut-out.

With regard to a secure coupling of the cartridge or sleeve to the syringe or to the adapter, the outer edge of the second opening of the cartridge or sleeve can be insertable into a nozzle of a syringe and/or into the nozzle of the adapter and the first opening can be fittable onto the nozzle of the same syringe and/or the same adapter. In each case, a secure coupling and formation of a fluidic connection between the holding region of the cartridge or sleeve and the syringe is ensured.

A provision and insertion of a transplant or implant into a body is further particularly reliable in that a good recognizability of the position of the transplant or implant in the cartridge or sleeve is provided. For this purpose, the cartridge or sleeve can be formed from a glass, for example, borosilicate glass, that is reflection-reduced on the inside and/or the outside. Undesirable mirroring and reflections which, for example, impair vision of the transplant or implant in the holding region of the cartridge or sleeve, for example, during an operation can thereby be prevented or at least as far as possible reduced.

A particularly reliable use of the device can be ensured by a ready-to-use set with a device as described above in a sterile packaging. Use is further simplified by such a ready-to-use set which also contains a syringe and/or a device into which a transplant or implant has already been introduced.

The designation cartridge or sleeve used in this document should be understood in the widest possible sense, specifically to describe a functional element which has a holding region and two openings at the end sides, each fluidically connected to the holding region, whereby the first opening has a larger diameter than the second opening and whereby both openings are couplable to the syringe to form a fluidic connection between the holding region and a syringe, in each case, directly or via a connecting apparatus.

Furthermore, the designation syringe is to be understood in the broadest sense, whereby any suitable syringe apparatus for drawing up and ejecting a liquid is meant.

Furthermore, the designations transplant and implant are to be understood in the broadest sense, specifically as an element, made of the most varied of materials, insertable in a human or animal body.

### BRIEF DESCRIPTION OF THE FIGURES

There is a wide variety of possibilities for configuring and developing the teaching of the present invention in an advantageous manner. For this purpose, reference is made firstly to the subordinate claims and, secondly, to the following explanation of a preferred exemplary embodiment of the device according to the invention on the basis of the drawing. In the context of the explanation of the preferred exemplary embodiment on the basis of the drawing, preferred embodiments and further developments of the teaching are also described in general. In the drawings:

FIGS. 1 to 5 are respectively schematic side views of an exemplary embodiment of a device according to the invention in different operating states, and

FIGS. 6 and 7 are enlarged perspective and side views of the device according to the invention of FIGS. 1 to 5 without the cartridge and the syringe.

### DETAILED DESCRIPTION OF VARIOUS EMBODIMENTS

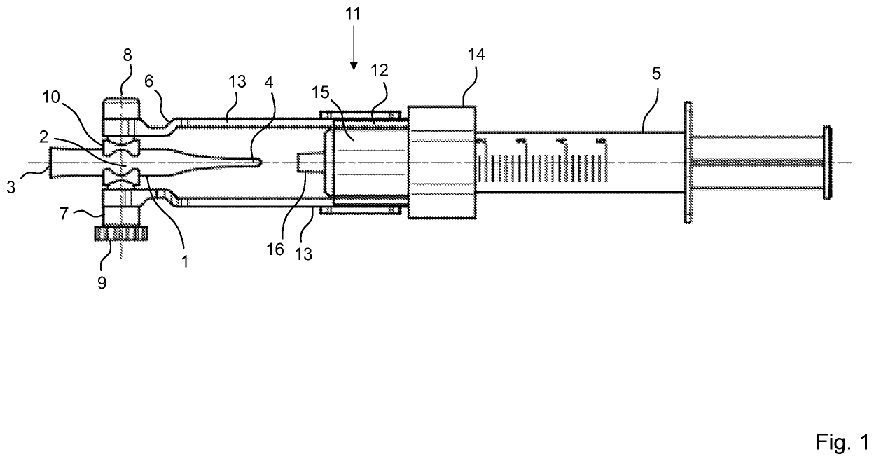

FIGS. 1 to 5 show an exemplary embodiment of a device according to the invention for providing and inserting a transplant or implant into a human or animal body in different operational states. The device preferably serves to carry out a DMEK operation. The device has a cartridge 1 which has a holding region 2 for the transplant or implant and two mutually opposite openings 3, 4 at the ends which are fluidically connected to the holding region 2. The first opening 3 has a larger diameter than the second opening 4. Both openings 3, 4 are couplable to a syringe 5, to form a fluidic connection between the holding region 2 and the syringe 5 directly or via a connecting apparatus.

With regard to a simple and secure provision and insertion of a transplant or implant into a human or animal body with simply designed means, the cartridge 1 is mounted in a holder 6, whereby the holder 6 has a pivoting apparatus 7 for pivoting the cartridge 1 about a pivot axis 8 into a respective coupling position of one of the two openings 3, 4 with the syringe 5.

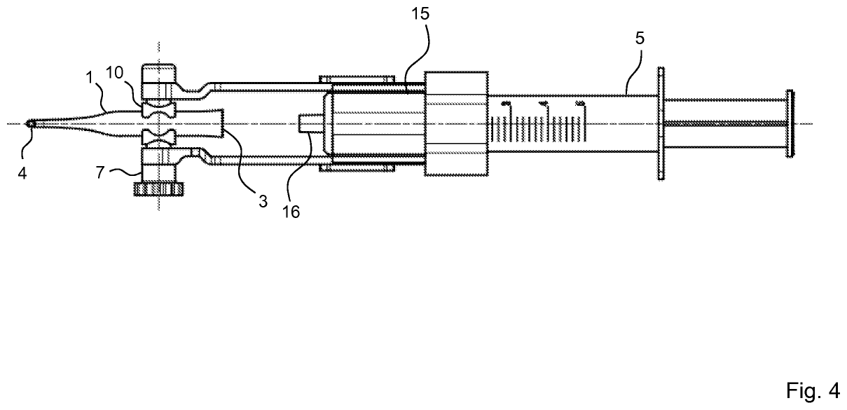

The pivoting apparatus 7 is configured for pivoting the cartridge 1 from a first coupling position for the first opening 3—see FIG. 4—by 180° about the pivot axis 8 into a second coupling position for the second opening 4—see FIG. 1—and vice versa. The pivoting apparatus 7 thereby has a snap-in locking device (not shown here) for locking, latching or detent of a pivoting movement of the cartridge 1 into the two coupling positions.

With regard to a simple actuation of the pivoting apparatus 7, the pivoting apparatus 7 has an actuating apparatus with a wheel 9 for pivoting the cartridge 1.

The pivoting apparatus 7 further has a fixing apparatus with a sleeve 10 for holding the cartridge 1 during the pivoting. By this means, a secure position of the cartridge 1 in the pivoting apparatus 7 is ensured.

For coupling the cartridge 1 to the syringe 5, the holder 6 has a holding apparatus 12 for a syringe 5, said holding apparatus being movable back and forth relative to the pivoting apparatus 7 by means of a guide 11. The guide 11 has a sled-like linear guide with two rails 13 extending parallel to one another. The holding apparatus 12 is movable between the rails 13. The rails 13 are connected to one another at their ends facing away from the pivoting apparatus 7 via a guide ring 14 for the syringe 5, in order to ensure a reliable guidance of the syringe 5, in particular, during its movement relative to the rails 13.

The holding apparatus 12 has an adapter 15 acting as a connecting apparatus with a cut-out for the syringe 5, whereby a nozzle 16 at the end extends out of the cut-out for coupling the cartridge 1. Alternatively, or in addition to such an adapter 15 with a cut-out and a nozzle 16, the holding apparatus 12 could have a fixing apparatus at least in parts encompassing the cylindrical region of the syringe 5, preferably in the form of a sleeve or bracket. In this case, a coupling could take place between the cartridge 1 and the nozzle of the syringe 5 in a direct manner, without the interposition of an adapter 15 with a nozzle 16.

The cartridge 1 is configured such that the outer edge of the second opening 4 is insertable into a nozzle of a syringe 5 or into the nozzle 16 of the adapter 15 and the first opening 3 is fitted onto the nozzle of the same syringe 5 or the same adapter 15.

In the exemplary embodiment shown here, the cartridge 1 is reflection-reduced both on its inside and on its outside. If relevant, in order to prevent mirror reflections, a reflection-reduction only on the outside is sufficient, since when the cartridge 1 is used, liquid is situated in the interior and prevents reflection.

With the exemplary embodiment shown here, a practically single-component system is realized which can be operated particularly reliably with two hands—or even, still reliably, one-handedly. For this purpose, the device with the pivoting apparatus 7 has a mechanism for pivoting and mutual docking of the cartridge 1 onto a syringe 5. The actual coupling between the cartridge 1 and the syringe 5 or adapter 15 takes place in the exemplary embodiment shown here with a linear movement between the cartridge 1 and the syringe 5 or adapter 15. By this means, either the drawing-in opening—first opening 3—or the inserting opening—second opening 4—can be fluidically connected to the syringe 5.

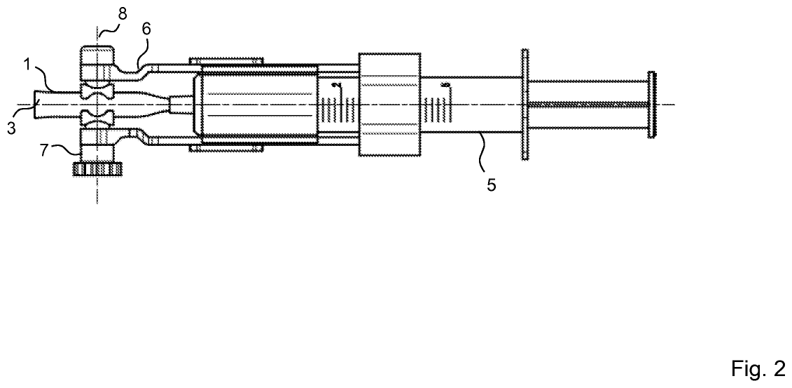

FIG. 1 shows the device in a state before a coupling of the cartridge 1 to the nozzle 16 with the adapter 15 for drawing-in a transplant or implant. Thereby, the cartridge 1 is pivoted with its second opening 4 toward the nozzle 16. By displacing the adapter 15 with the syringe 5 arranged therein along the rails 13, the coupling is performed. Thereby—as shown in FIG. 2—the second opening 4 is inserted into the nozzle 16, whereby the adapter 15 has been guided with the syringe 5 along the rails 13 in the direction of the pivoting apparatus 7, or the pivoting apparatus 7 has been guided with the cartridge 1 in the direction of the adapter 15. Therefore, FIG. 2 shows the operating state of the device in which a transplant or implant, for example, a DMEK lamella can be drawn into the holding region 2.

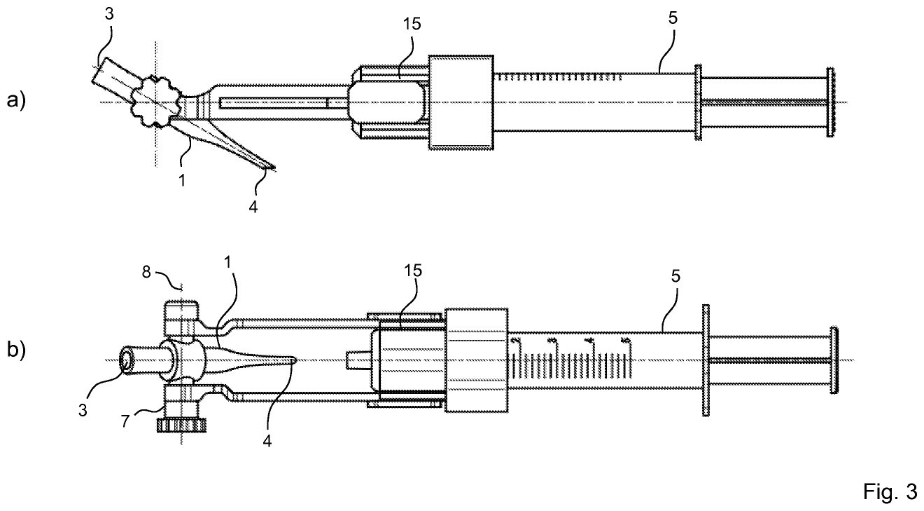

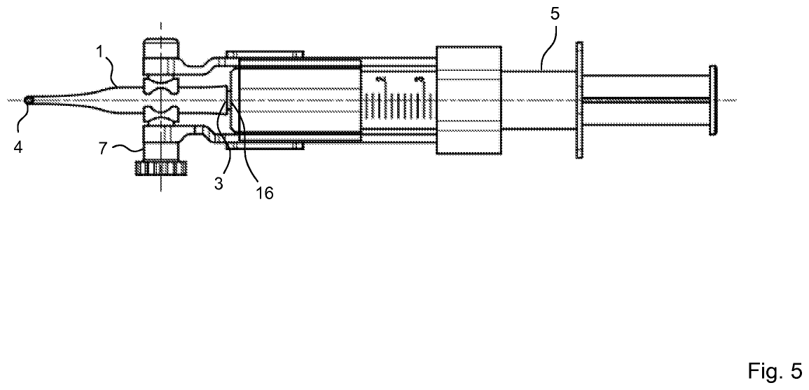

FIG. 3 shows, in two side views rotated by 90° relative to one another, an operating state of the device with pivot apparatuses 7 and adapter 15 moved away from one another. In this state, a free pivoting of the cartridge 1 about the pivot axis 8 is possible in order to prepare a coupling between the nozzle 16 and the first opening 3. This pivoting process is completed in the operating state shown in FIG. 4. The pivoting apparatus 7 with the cartridge 1 and the adapter 15 with the syringe 5 can now be moved toward one another in order to reach the operating state shown in FIG. 5 with the first opening 3 pushed onto the nozzle 16. In the state shown in FIG. 5, an insertion of a transplant or implant from the holding region 2 through the second opening 4 into a body is possible.

In the device according to the invention, a connection between a cartridge 1 or sleeve and a suitable syringe 5 is enabled via a holder 6 which can be operated in a simple manner. The conventional connecting possibility by means of a flexible connecting hose does not ensure this reliable and simple operability of the system made from the cartridge 1 or sleeve and a suitable syringe 5. With the device according to the invention, a simple rotation of the cartridge 1 between the injection or the aspiration side is enabled in order to inject or aspirate a transplant or implant. In an advantageous manner, a locking, latching or detent of a pivot movement of the cartridge 1 or sleeve relative to the holder 6 into the two coupling positions or into end positions of the pivoting (180°) can be realized in order to ensure a reliable fitting or insertion of the cartridge 1 onto or into a nozzle of a syringe 5 or nozzle 16 of an adapter 15 without misalignment. The fitting on or inserting can take place, controlled by means of a sled-like linear guide or a bayonet-like rotary guide by means of a relative movement between the cartridge 1 and the syringe 5 or the adapter 15. Both the openings 3 and 4 of the cartridge 1 can be coupled in a sealed manner to the nozzle of a syringe 5 or the nozzle 16 of an adapter 15.

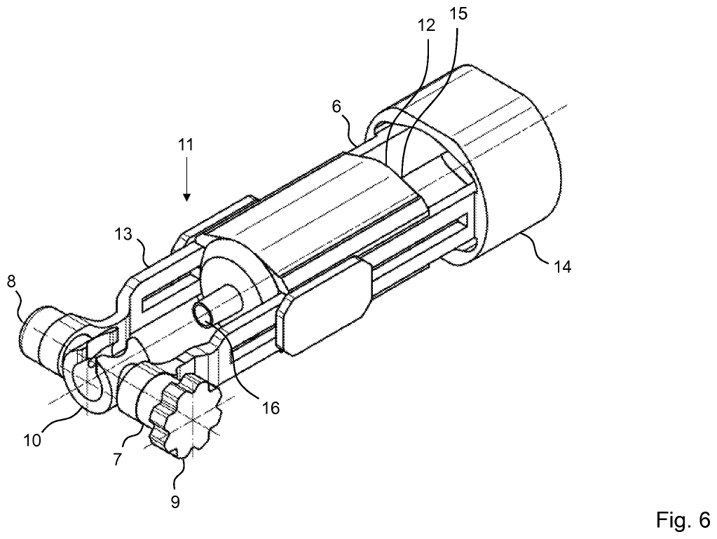

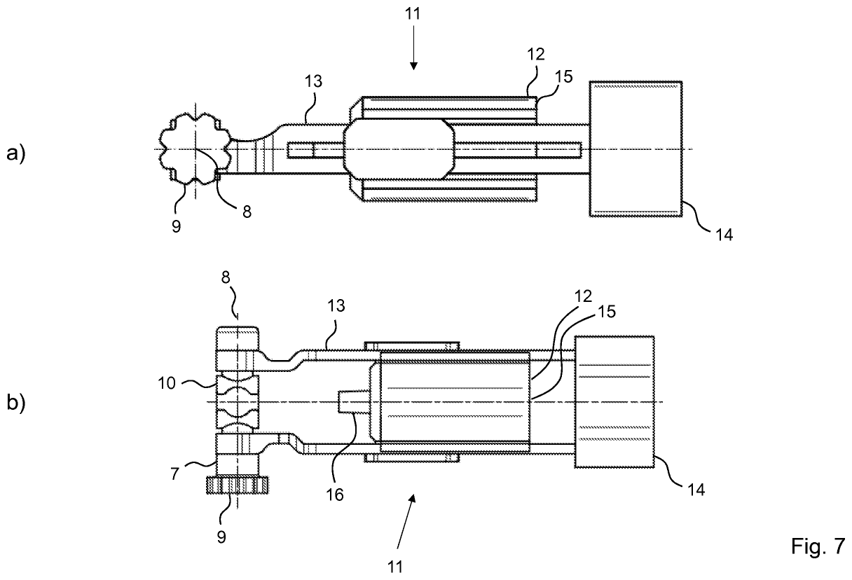

FIGS. 6 and 7 show, in an enlarged perspective view (FIG. 6) and rotated by 90° thereto, in a side view (FIG. 7), the device according to the invention of FIGS. 1 to 5, whereby for the sake of clarity, the cartridge 1 and the syringe 5 are omitted. The reference signs used in FIGS. 6 and 7 correspond to the reference signs shown in FIGS. 1 to 5 and described in relation thereto.

To reiterate, in a manner according to the invention, it was firstly discovered that a simple and reliable introduction of a transplant or implant into a human or animal body does not necessarily have to take place with the conventionally usual system consisting of three components in the form of a cartridge or sleeve, a connecting hose and a syringe. In a further manner according to the invention, it was then discovered that with skillful mounting of the cartridge or sleeve in a holder, the above-mentioned object is achieved in a surprising manner. For this purpose, according to the invention, the cartridge or sleeve is mounted quite specifically in a holder, whereby the holder has a pivoting apparatus for pivoting the cartridge or sleeve about a pivot axis. The pivoting of the cartridge or sleeve takes place in such a way that the cartridge or sleeve is brought into a—directly or, via a connecting apparatus, indirectly—coupling position of at least one of the two openings with the syringe. The coupling of the cartridge or sleeve in a suitable coupling position is very significantly simplified by the mounting according to the invention of the cartridge or sleeve in a holder with a pivoting apparatus, whereby the pivoting of the cartridge or sleeve is typically possible—even one-handedly—by grasping the holder and actuating the pivoting apparatus. Following the desired pivoting of the cartridge or sleeve into a suitable coupling position, a coupling with the syringe—directly, or indirectly via a connecting apparatus—can take place in a simple and reliable manner. Incorrect operations are practically precluded.

Consequently, with the device according to the invention and also a corresponding ready-to-use set with such a device, a simple and reliable provision and insertion of a transplant or implant into a human or animal body is enabled with simply designed means.

With regard to a particularly simple and reliable provision and insertion of a transplant or implant, the pivoting apparatus can be configured for pivoting the cartridge or sleeve from a first coupling position for the first opening, depending on the geometry of the cartridge or sleeve—with a straight, elongate geometry (both openings diametrically opposite)—preferably by 180°, about the pivot axis into a second coupling position for the second opening and vice versa. In another embodiment of the device, a pivoting by 120° or another angle could be sufficient to change between the first and second coupling position. The pivoting apparatus thereby provides a pivoting possibility for the cartridge or sleeve between the two utilization positions of the cartridge or sleeve, firstly, for drawing in a transplant or implant and, secondly, for injecting or inserting a transplant or implant. Thereby, both the provision and also the insertion of a transplant or implant is enabled, depending on the coupling position selected for the first or second opening.

With regard to a further simplification and increase in the reliability on provision and insertion of a transplant or implant, the pivoting apparatus can have a snap-in locking device for locking, latching or detent of a pivoting movement of the cartridge or sleeve in at least one of the two coupling positions. As soon as the cartridge or sleeve has reached at least one of the two coupling positions by means of the pivoting, the locking, latching or detent of the cartridge or sleeve takes place for fixing the cartridge or sleeve in the first and/or second coupling position. The snap-in locking device can have suitable grooves in conjunction with locking noses—each, for example, in surfaces or components of the pivoting apparatus sliding over one another during the pivoting—which, on reaching the first and/or second coupling position, engage with one another for locking, latching or detent of the pivoting movement.

With regard to a simple achievement of at least one of the two coupling positions of the cartridge or sleeve, the pivoting apparatus can have an actuating apparatus for pivoting the cartridge or sleeve. In a particularly simple manner, such an actuating apparatus can have a lever or a wheel.

In order to ensure a reliable mounting of the cartridge or sleeve during use of the device, the pivoting apparatus can have a fixing apparatus for holding the cartridge or sleeve during the pivoting. By this means, a reliable positioning of the cartridge or sleeve in a desired coupling position is enabled with a high degree of safety. Such a fixing apparatus can have, for example, a sleeve or bracket for preferably completely grasping the cartridge or sleeve. By this means, a particularly secure fixing of the cartridge or sleeve during the pivoting is achievable.

Furthermore, with regard to a particularly simple and reliable provision and insertion of a transplant or implant, the holder can have a holding apparatus for a syringe, said holding apparatus being movable back and forth relative to the pivoting apparatus by means of a guide. After reaching a desired coupling position through the pivoting of the cartridge or sleeve by means of the pivoting apparatus, the holding apparatus for a syringe can be moved toward the pivoting apparatus, so that the coupling between the cartridge or sleeve and the syringe can be carried out—with this axial movement process, also, a locking or latching into the end positions can be realized. During the movement of the holding apparatus relative to the pivoting apparatus, the direct or indirect coupling takes place between the cartridge or sleeve with the syringe or the formation of the fluidic connection between the holding region of the cartridge or sleeve and the syringe. In order to swap the coupling position of the cartridge or sleeve, the holding apparatus can be moved away relative to the pivoting apparatus by means of the guide, in order to enable a free pivoting of the cartridge or sleeve in the state uncoupled from the syringe. In this way, swapping can take place between the coupling of the syringe with the first opening and with the second opening of the cartridge or sleeve. It should be noted that the movement between the pivoting apparatus and the holding apparatus involves a movement of these two components relative to one another. Both components are associated with the holder or arranged thereon and either the pivoting apparatus can be moved in the direction of the holding apparatus or away therefrom, or the holding apparatus can be moved toward or away from the pivoting apparatus or both the pivoting apparatus and also the holding apparatus can be moved toward or away from one another, in order to realize the desired relative movement.

In an advantageous manner, the guide can have a preferably sled-like linear guide. This linear guide can have at least one rail configured fixed to the pivoting apparatus or to the holding apparatus, along which a sliding element is displaceable back and forth, wherein the sliding element can be coupled to syringe or the pivoting apparatus in order to be able to move the syringe relative to the pivoting apparatus and thus relative to the cartridge or sleeve. The sliding element can be coupled—if the at least one rail is configured fixed to the pivoting apparatus—to the holding apparatus for the syringe or such a holding apparatus for a syringe. In this way, a particularly compact configuration of the device can be realized. Alternatively, the guide can have a preferably bayonet-like twist guide which ensures the relative movement between the pivoting apparatus and the holding apparatus for the syringe. Such a twist guide provides, firstly, a linear component of the relative movement between the pivoting apparatus and the holding apparatus and, secondly, a rotation component about the direction of the linear component. Preferably, one end position of this twist-slide guide forms a coupling position between the cartridge or sleeve, and another end position forms a non-coupled position in which the cartridge or sleeve can be pivoted by means of the pivoting apparatus. It should again be noted at this point that a coupling can take place between the syringe and the cartridge or sleeve, both directly as well as via a connecting apparatus. In this regard, these two possibilities of the coupling—direct or indirect—are always included wherever a coupling between the cartridge or the sleeve and the syringe is mentioned in this document.

Furthermore, with regard to a particularly simple and reliable provision and insertion of a transplant or implant into a body, the holding apparatus can have a fixing apparatus at least in parts encompassing a cylindrical region of the syringe, preferably a sleeve or bracket. By means of such a fixing apparatus, a secure coupling of a syringe to the holder of the device is enabled. A reliable overall function of the device is, in particular, thereby ensured.

Alternatively or in addition to such a fixing apparatus, the holding apparatus can have an adapter operating as a connecting apparatus with a cut-out or a coupling region for the syringe and preferably a nozzle extending at the end side out of the cut-out or out of the coupling apparatus for coupling to the cartridge or sleeve. In this case, the coupling does not take place directly between the syringe and the cartridge or sleeve, but rather indirectly via an adapter acting as a connecting apparatus. For the secure coupling of a syringe, the adapter has a cut-out or a coupling region for the syringe. In order to ensure a fluidic connection between the cut-out region of the cartridge or sleeve and the syringe, the adapter preferably has a nozzle extending out of the cut-out or out of the coupling region, with which the cartridge or sleeve can be coupled. In this case, the syringe is insertable into the cut-out, whereby the cut-out can have a hollow space that is substantially cylindrical or adapted to the external form of the syringe, in order to ensure a secure and exactly fitting holding of the syringe in the cut-out.

With regard to a secure coupling of the cartridge or sleeve to the syringe or to the adapter, the outer edge of the second opening of the cartridge or sleeve can be insertable into a nozzle of a syringe and/or into the nozzle of the adapter and the first opening can be fittable onto the nozzle of the same syringe and/or the same adapter. In each case, a secure coupling and formation of a fluidic connection between the holding region of the cartridge or sleeve and the syringe is ensured.

A provision and insertion of a transplant or implant into a body is further particularly reliable in that a good recognizability of the position of the transplant or implant in the cartridge or sleeve is provided. For this purpose, the cartridge or sleeve can be formed from a glass, for example, borosilicate glass, that is reflection-reduced on the inside and/or the outside. Undesirable mirroring and reflections which, for example, impair vision of the transplant or implant in the holding region of the cartridge or sleeve, for example, during an operation can thereby be prevented or at least as far as possible reduced.

A particularly reliable use of the device can be ensured by a ready-to-use set with a device as described above in a sterile packaging. Use is further simplified by such a ready-to-use set which also contains a syringe and/or a device into which a transplant or implant has already been introduced.

Finally, reference is explicitly made to the fact that the embodiment described above serves merely for explanation of the claimed teaching, but without restricting it to this embodiment.

1-12. (canceled)

13. A device for providing and inserting a transplant or an implant into a human or animal body, in particular for ophthalmological procedures, preferably for carrying out a lamellar keratoplasty, for example, a DMEK operation, the device comprising a cartridge (1) or sleeve which has a holding region (2) for the transplant or implant and two opposite end openings (3, 4), each fluidically coupled to the holding region (2), wherein the first opening (3) has a larger diameter than the second opening (4) and wherein both openings (3, 4) are couplable to a syringe (5) directly or by means of a connecting apparatus to form a fluidic connection between the holding region (2) and the syringe (5), and wherein the cartridge (1) or sleeve is mounted in a holder (6) and the holder (6) has a pivoting apparatus (7) for pivoting the cartridge (1) or sleeve about a pivot axis (8) into a coupling position of at least one of the two openings (3, 4) with the syringe (5).

14. The device according to claim 13, wherein the pivoting apparatus (7) is configured for pivoting the cartridge (1) or sleeve from a first coupling position for the first opening (3) about the pivot axis (8) into a second coupling position for the second opening (4) and vice versa.

15. The device according to claim 14, wherein the pivoting is 180° about the pivot axis (8).

16. The device according to claim 14, wherein preferably the pivoting apparatus (7) has a snap-in locking device for locking, latching or detent of a pivoting movement of the cartridge (1) or sleeve in at least one of the two coupling positions

17. The device according to claim 13, wherein the pivoting apparatus (7) has an actuating apparatus for pivoting the cartridge (1) or sleeve.

18. The device according to claim 17, wherein the actuating apparatus has a lever or a wheel (9).

19. The device according to claim 13, wherein the pivoting apparatus (7) has a fixing apparatus for holding the cartridge (1) or sleeve during the pivoting.

20. The device according to claim 19, wherein the fixing apparatus has a sleeve (10) or a bracket.

21. The device according to claim 13, wherein the holder (6) has a holding apparatus (12) for a syringe (5), said holding apparatus being movable back and forth relative to the pivoting apparatus (7) by means of a guide (11).

22. The device according to claim 21, wherein the guide (11) has a sled-like linear guide or a bayonet-like twist guide.

23. The device according to claim 21, wherein the holding apparatus (12) has a fixing apparatus at least in part encompassing a cylindrical region of the syringe (5), preferably a sleeve or bracket.

24. The device according to claim 23, wherein the cylindrical region of the syringe (5) is a sleeve or a bracket.

25. The device according to claim 21, wherein the holding apparatus (12) has an adapter (15) acting as a connecting apparatus with a cut-out or a coupling region for the syringe (5).

26. The device according to claim 25, wherein the holding apparatus (12) has a nozzle (16) extending at the end out of the cut-out or out of the coupling region for coupling the cartridge (1) or sleeve.

27. The device according to claim 13, wherein the outer edge of the second opening (4) is insertable into a nozzle of a syringe (5) and/or into the nozzle (16) of the adapter (15) and the first opening (3) can be fitted onto the nozzle (16) of the same syringe (5) and/or the same adapter (15).

28. The device according to claim 13, wherein the cartridge (1) or sleeve is formed from a glass that is reflection-reduced on at least one of an inside or an outside.

29. The device according to claim 28, wherein the glass is borosilicate glass.

30. A ready-to-use set comprising a device according to claim 13, in a sterile packaging.

31. The ready-to-use set according to claim 30, wherein the set additionally includes a syringe (5).

32. The ready-to-use set according to claim 30, wherein the device contains a transplant or implant.
